(19) United States
(12) Patent Application Publication
(10) Pub. No.: US 2007/0243158 A1
(43) Pub. Date: Oct. 18, 2007

(54) SKIN CARE COMPOSITIONS AND TREATMENTS

(75) Inventors: Vincent Ronfard, West Newton, MA (US); Alan W. Tuck, Sherborne, MA (US); Leon M. Wilkins, North Andover, MA (US)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(73) Assignee: ORGANOGENESIS, INC., Canton, MA (US)

(21) Appl. No.: 11/761,893

(22) Filed: Jun. 12, 2007

Related U.S. Application Data
(63) Continuation of application No. 10/149,949, filed on Jan. 27, 2003.
(60) Provisional application No. 60/151,463, filed on Aug. 30, 1999.
(60) Provisional application No. 60/150,221, filed on Aug. 23, 1999.

(51) Int. Cl. A61Q 19/08; A61K 45/00; C12N 5/06; C12P 1/00

(52) U.S. Cl. 424/85.1; 435/404; 435/41

(57) ABSTRACT

The invention is directed to compositions containing growth agents synthesized from cultured cells from skin. Skin cells such as keratinocytes and dermal fibroblasts are cultured in vitro in cell medium and in the course of culture the cultured cells synthesize and secrete agents into the cell medium. The medium containing agents are collected and incorporated into pharmaceutical or cosmetic preparations to treat an individual. The preparation is applied and has a rejuvenating effect on the cells and tissue.

### 1. FIELD OF THE INVENTION

The field of the invention is cell culture and medical biotechnology, particularly compositions containing cultured skin agents synthesized from cultured cells from skin. Skin cells such as keratinocytes and dermal fibroblasts are cultured in vitro in cell medium and in the course of culture the cultured cells synthesize and secrete agents into the cell medium. The medium containing agents are collected and incorporated into topical preparations to treat an individual. The preparation is applied to a patient's skin and has a rejuvenating effect on the cells and tissue.

### 2. BACKGROUND OF THE INVENTION

As skin ages, dryness and loss of elasticity become more prevalent. In addition, exposure to sun, wind, pollution and other external irritants and environmental stresses can aggravate skin aging. To improve skin appearance, a number of skin-care products and skin care treatments have been developed. In addition, a variety of medical treatments have been developed to treat chronic skin problems, such as acne, precancerous lesions, scars, pigmentation disorders, wrinkles and the like. There are several recent topically applied skin care products and skin care treatments that are currently being used or being researched. For example, vitamin-A (retinol) and vitamin-A derivatives, called retinoids, are topical treatments believed to work by loosening the top layer of skin and encouraging cellular turnover. Topical applications of vitamin C (ascorbic acid), which neutralizes free radicals, are used to heal skin and reduce the appearance of fine lines and wrinkles. Vitamin K is topically applied to help heal broken blood vessels, spider veins, bruises, under-eye circles and blotchy red skin. Alpha-hydroxy acids (AHAs) and beta-hydroxy acids (BHAs) are topical exfoliants that improve skin vibrancy and help prevent acne. Topical applications of epidermal growth factor (EGF) may improve skin function and create an overall more youthful appearance.

As an alternative to plastic or facial surgery to make skin appear youthful, many patients are opting for skin resurfacing treatments that are not as invasive as surgical procedures. These procedures include laser peels, chemical peels, dermabrasion and dermaplaning. All skin resurfacing treatments work essentially the same way. First, the outer layers of damaged skin are stripped away to a degree that levels the depth of wrinkles and scars to the surrounding skin. For superficial or medium resurfacing, the layers of skin tissue removed can be limited to the epidermis and papillary dermis. For deeper resurfacing, the upper levels of the reticular dermis can also be removed. Varied penetration allows treatment of specific spots or wrinkles. In the time after treatment, as new cells multiply and migrate into the resurfaced area during the healing process, a smoother, tighter, younger-looking skin surface appears. During the healing process, skin care products are applied to the treated area to enhance and accelerate skin healing.

It is a continuing goal of both the cosmetic industries and the pharmaceutical companies to develop skin care products and skin care treatments to improve skin appearance and to improve the healing process of damaged skin.

### SUMMARY OF THE INVENTION

This invention is based on the discovery that the conditioned cell medium can be made into a composition or preparation for use in topically treating skin. The composition of this invention is a conditioned medium containing one or more cultured skin agents synthesized and secreted from cultured skin cells for use as a pharmaceutical preparation or as a skin care product.

As a pharmaceutical preparation, the product is topically applied to treat skin conditions, such as promoting wound healing. The topical composition can be combined with any appropriate pharmaceutically acceptable carrier.

As a skin care product, or as a skin care treatment, the product is topically applied to the skin to improve the appearance of the skin in an amount sufficient to increase cell proliferation and generation and to decrease cell senescence.

The invention is also directed to a method for producing a composition or a preparation containing a conditioned cell medium containing one or more cultured skin agents produced by cultured skin cells. The method includes culturing skin cells, either keratinocytes or fibroblasts, or preferably co-culturing both cell types, in a nutrient containing medium to grow the skin cells and then inducing the cells to synthesize and secrete one or more cytokines into the medium. The thus produced conditioned cell medium, now containing one or more cultured skin agents, is separated from the cultured skin cells and used to produce a composition or preparation for topical administration to the skin.

### DESCRIPTION OF THE FIGURES

FIG. 1 depicts an apparatus for forming a skin construct that produces cytokines and deposits them into the surrounding medium to condition it.

FIG. 2 is a graph showing the effect of conditioned medium (ACM) on keratinocyte colony size.

FIG. 3 is a graph showing the effect of conditioned medium (ACM) on keratinocyte proliferation.

FIG. 4 is a graph showing the effect of conditioned medium (ACM) on keratinocyte migration on fibrin.

FIG. 5 is a graph showing the effect of conditioned medium (ACM) on keratinocyte helix turns in migration along a fibrin substrate.

FIG. 6 is a graph showing the effect of conditioned medium (ACM) on endothelial cell proliferation.

FIG. 7 is a graph showing the effect of conditioned medium (ACM) on smooth muscle cell proliferation.

FIG. 8 is a graph showing the effect of conditioned medium (ACM) on fibroblast proliferation.

FIG. 9 is a graph showing the characterization of conditioned medium (ACM) cytokines in conditioned medium, the cotton pad, and skin construct cell extract.

FIG. 10 is a graph demonstrating that the effect of the conditioned medium (ACM) is independent of the EGF-receptor pathway.

### DETAILED DESCRIPTION OF THE INVENTION

The invention is directed to conditioned medium composition containing cultured skin agents produced from cultured cells of skin such as dermal fibroblasts and epidermal cells. The cultured skin agents in the conditioned medium are biologically active molecules that are used to formulate pharmaceutical, cosmetic, and wound healing preparations.

When cultured skin agents from the cells are used in the field of cosmetic formulations, they benefit the consumer for overall skin rejuvenation, including the appearance of enhanced pliability, softness and elasticity; wrinkle reduction; reduced evidence of the aging process and repair of the skin. The cultured skin agents are absorbed by the skin and initiate proliferation and generation of new skin cells, keratinocytes and fibroblasts, the key cell types found in skin, and decrease cell senescence of skin cells to hinder, halt, or reverse skin wrinkling.

As a pharmaceutical preparation, the cultured skin agents in the conditioned media are used for the enhancement of the healing process after second degree burns, skin treatments, moisture retention; pain reduction; soothingness, and establishment of more complete healing with new skin faster after dermabrasion, dermaplaning, exfoliation, chemical peel, laser treatment, sunburn, windburn, irradiation burns, skin treatments, blistering, spa treatments, and other procedures or events that cause skin trauma. Pharmaceutical preparations containing these cytokines may similarly be used to treat surfaces of the mucous membranes after surgery or injury.

In wound healing preparations, a preparation containing cultured skin agents from conditioned media is used by directly applying the preparation to the wound bed or by incorporation into a wound dressing. The preparation may be used as an adjunct with grafts, such as an autograft (skin removed from a patient and reapplied elsewhere on the same patient) or a cultured skin construct by coating the graft surface, the entire graft or the wound bed with the preparation. When used as an adjunct in wound healing, the cultured skin agents contained in to the wound healing preparation generally increase and improve wound closure by inducing keratinocyte and fibroblast proliferation and generation and granulation tissue and blood vessel formation.

Conditioned medium means medium that has contacted a tissue culture and has been used by the cells of the tissue culture as a source of nutrients, vitamins, hormones, and inorganic compounds and salts and by having contacted the tissue culture, now has added cell products, or “cultured skin agents”, such as cytokines, proteins, extracellular matrix components, or any combination thereof, synthesized and secreted by the cells into the medium. Conditioning is the act of the cells' synthesis and secretion of cytokines, proteins and extracellular matrix components, into fresh medium upon contact, exposure, exchange and interaction with between the cells and the medium for a time to condition the medium. The then conditioned medium is removed from the culture apparatus containing the skin construct in culture and collected for purification of its cultured skin agents or used whole or in part as a pharmaceutical, cosmetic or wound healing composition or for use in cell culture, in vitro.

Cytokines are proteins that exert changes in the function or activity of a cell such as differentiation, proliferation, secretion or motility. Growth factors are a subset of cytokines that are also proteins that cause changes in functions or activities that promote or inhibit cellular growth, proliferation, migration, or other related cellular events. Chemokines are another subset of cytokines that attract and guide T-cells, B-cells, and other chemokine-responsive cells to specific tissues in the body. Lymphokines are still another subset of cytokines involved in immune response. As used herein, the term, “cytokines”, includes cytokines, including growth factors, chemokines and lymphokines, and are not limited to their normal structure and function, but may also include their naturally occurring variants and hybrids.

Throughout their fabrication and when fully formed, cultured skin constructs contain living cells that synthesize and secrete an array of cytokines and other substances into the matrix of the construct and into the medium bathing the construct. The cultured cells in the cultured skin constructs typically consist of dermal fibroblasts and epidermal cells, epidermal cells are also referred to as keratinocytes. In the process of fabricating and culturing a bilayer skin construct, the epidermal and dermal tissue layers provide a tissue-like environment, an organized co-culture incorporating an extracellular matrix, for cell-cell and cell-matrix interactions similar to those that occur in native mammalian and human skin. These interactions in the developing construct allow for a wide profile of cytokine expression and secretion to the media to induce other cells in the culture to perform functions of extracellular matrix development, basement membrane production, and cell proliferation and differentiation.

Cytokines that are produced by cultured skin constructs that are a feature of this invention include, but are not limited to: basic fibroblast growth factor (bFGF); epidermal growth factor (EGF); keratinocyte growth factor (KGF), transforming growth factor alpha (TGFα); transforming growth factor beta (TGFβ), including transforming growth factor beta-1 (TGFβ1) and transforming growth factor beta-2 (TGFβ2); granulatory colony stimulating factor (GCSF); insulin-like growth factor (IGF); vascular endothelial growth factor (VEGF), and tumor necrosis factor (TNF). In the chemokine subset, interleukins affect cell apoptosis. A number of interleukins including interleukin-1, interleukin-6, interleukin-8, interleukin-11 are also synthesized by the developing skin construct and are also a feature of this invention. It should be noted that the aforementioned terms in parentheticals are abbreviations commonly known and used in the art for the formal nomenclature preceding them.

Preferably, the conditioned media of the invention are produced by cultured cells of skin cells; keratinocytes, dermal fibroblasts, or both, more preferably when the cells are cultured together as a co-culture of both keratinocytes and dermal fibroblasts. The conditioned media of the invention are most preferably produced when the co-culture is a cultured skin construct having at least a dermal layer and an epidermal layer arranged in orientation similar to native skin. Dermal layers comprise fibroblast cells, preferably of dermal origin and extracellular matrix, primarily of collagen. It will be appreciated by the skilled artisan that the cultured skin construct may contain, by either intentional addition or with continued culture of fibroblasts from primary sources, other cells found in skin and other extracellular matrix components.

Preferred cell types for use in this invention are derived from mesenchyme. More preferred cell types are fibroblasts, stromal cells, and other supporting connective tissue cells, or, as in the most preferred embodiment, human dermal fibroblasts. Human fibroblast cell strains can be derived from a number of sources, including, but not limited to neonate male foreskin, dermis, tendon, lung, umbilical cords, cartilage, urethra, corneal stroma, oral mucosa, and intestine. The human cells may include but need not be limited to: fibroblasts, smooth muscle cells, chondrocytes and other connective tissue cells of mesenchymal origin. It is preferred, but not required, that the origin of the matrix-producing cell used in the production of a tissue construct be derived from a tissue type that it is to resemble or mimic after employing the culturing methods of the invention. For instance, a multilayer sheet construct is cultured with fibroblasts to form a living connective tissue construct; or myoblasts, for a skeletal muscle construct. More than one cell type can be used to fabricate a tissue construct. Cell donors may vary in development and age. Cells may be derived from donor tissues of embryos, neonates, or older individuals including adults. Embryonic progenitor cells such as mesenchymal stem cells may be used in the invention and induced to differentiate to develop into the desired tissue.

Although human cells are preferred for use in the invention, the cells to be used in the method of the are not limited to cells from human sources. Cells from other mammalian species including, but not limited to, equine, canine, porcine, bovine, feline, caprine, and ovine sources may be used. Murine cells, and other cells from rodent sources, may also be used. In addition, genetically engineered cells that are spontaneously, chemically or virally transfected may also be used in this invention. For those embodiments that incorporate more than one cell type, mixtures of normal and genetically modified or transfected cells may be used and mixtures of cells of two or more species or tissue sources may be used, or both.

Recombinant or genetically-engineered cells may be used in the production of the tissue construct to create a tissue construct that acts as a drug delivery graft for a patient needing increased levels of natural cell products or treatment with a therapeutic. The cells may produce recombinant cell products, growth factors, hormones, peptides or proteins for a continuous amount of time or as needed when biologically, chemically, or thermally signaled due to the conditions present in culture. Cells may also be genetically engineered to express cytokines, proteins or different types of extracellular matrix components which are either ‘normal’ but expressed at high levels or modified in some way to make a cell products that are therapeutically advantageous for improved wound healing, facilitated or directed neovascularization. These procedures are generally known in the art, and are described in Sambrook et al, Molecular Cloning, A Laboratory Manual, Cold Spring Harbor Press, Cold Spring Harbor, N.Y. (1989), incorporated herein by reference. All of the above-mentioned types of cells may be used in this invention for the production of a cultured skin construct that will synthesize the conditioned media containing cytokines.

While collagen is the most preferred extracellular matrix composition for use in the production of skin equivalents that produce and secrete cytokines and other cultured skin agents to condition the culture media, other extracellular matrix components may be used. These extracellular matrix components may be used alone or, preferably, be included with the collagen to mimic native dermal matrix. These extracellular matrix components may include: other collagens, both fibrillar and non-fibrillar collagen from the collagen family such as collagen types II, III, IV, V, VI, VII, VIII, IX, X, XI, XII, XIII, XIV, XV, XVI, XVII, XVI, XIX, other matrix proteins that may include, but are not limited to elastin, proteoglycans such as decorin or biglycan, or glycoproteins such as tenascin, vitronectin, fibronectin, laminin, thrombospondin I, and glycosaminoglycans (GAG) such as hyaluronic acid (HA). The dermal matrix may vary in composition and structure. Collagen sponges, biocompatible, bioremodelable, decellularized dermis, or collagen gels. Rather than provide extracellular matrix components to the dermal cells, they can be cultured on biodegradable mesh members (such as nylon or polygalactin (PGA)) to provide a culture support and cultured to produce extracellular matrix until the cells and their matrix envelope the support. In the preferred embodiment, the dermal layer is a contracted collagen gel, contracted by fibroblasts such as those described in U.S. Pat. No. 4,485,096 to Bell, incorporated herein by reference. In a more preferred embodiment, the contracted collagen gel is disposed on a bulk acellular collagen layer on a porous membrane to anchor the gel to the membrane and to prevent excessive radial contraction of the gel. Methods for incorporating a bulk acellular collagen layer are described in U.S. Pat. No. 5,536,656 to Kemp, et al., in Wilkins, L. M., et al. Development of a Bilayered Living Skin Construct for Clinical Applications. Biotechnology and Bioengineering, vol. 43, pp. 747-756 (1994), and in Parenteau, N. L. Skin equivalents. In: I. Leigh and F. Watt (eds.), The Keratinocyte Handbook. Cambridge University Press, London (1994), the disclosures of which are incorporated herein by reference.

Both the tissue equivalent and the acellular, hydrated collagen gel in accordance with the present invention may be prepared using collagen derived from skin and tendon, including rat tail tendon, calf skin collagen, and calf extensor tendon. Other sources of collagen would be suitable. A particularly preferred collagen composition derived from calf common digital extensor tendon and methods of deriving such collagen compositions are disclosed in U.S. Pat. No. 5,106,949 to Kemp, the disclosure of which is incorporated herein by reference.

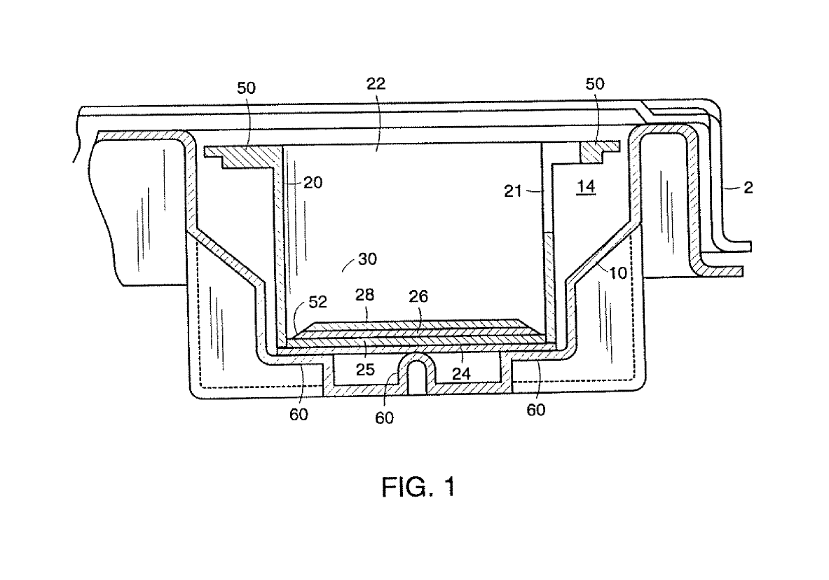

In one method of the present invention, referring to FIG. 1, an acellular, hydrated collagen gel 25 is prepared from a collagen composition comprising collagen at about 0.5 to 2.0 mg/ml, preferably about 0.9 to 1.1 mg/ml and nutrient media. This collagen composition is added to the inner container 20 and maintained under conditions which permit the collagen composition to set and form an acellular, hydrated collagen gel of suitable dimensions, typically about 1 to 5 mm thick, a preferred thickness range being about 2 to about 3 mm. An acellular, hydrated collagen gel 25 is preferably thick enough so that a portion remains acellular as cells migrate from the tissue equivalent into an acellular, hydrated collagen gel and thin enough so that the tissue equivalent is not undesirably removed from the nutrient source provided in outer container 10.

A dermal equivalent is next cast on an acellular, hydrated collagen gel using procedures in accordance with the aforementioned Patents and as described hereinafter. A casting mixture containing collagen and fibroblasts is added to inner container 20 over an acellular, hydrated collagen gel 25 and maintained under conditions that enable the tissue equivalent to form. As the tissue equivalent forms on an acellular, hydrated collagen gel 25, it contracts radially.

Typically, the sides of the dermal layer 26 slope towards the outer periphery of hydrated collagen gel 25 to form a mesa as shown in FIG. 1 at 52. The dermal layer 26 is now seeded with epithelial cells to form the epidermal layer 28. The epidermal cells are seeded in culture medium at a concentration of between about 0.3×10⁶ to about 30×10⁶ cells/ml. The volume of epidermal cells seeded will depend upon the size of the mesa.

The concentration of collagen, the number of cells and the volume of the casting mixture can be controlled to optimize the diameter and thickness of the living tissue equivalent. The casting mixture comprises cells at a concentration of about 1.25×10⁴ to about 5×10⁴ cells/ml and collagen at about 0.5 to about 2.0 mg/ml in a nutrient medium. A preferred cell concentration is about 2.5×10⁴ cells/ml. It has been found that the ratio of the volume of the casting mixture for the tissue equivalent to the volume of the casting mixture for the acellular, hydrated collagen gel has an effect upon cell viability and differentiation. Useful ratios, volume to volume (v/v), of tissue equivalent casting mixture to collagen gel casting mixture are about 3:1 to 1:3. A preferred ratio wherein the cell concentration in the collagen lattice is at about 2.5×10⁴ cells/ml is 3:1.

The cultures are maintained in an incubator to ensure sufficient environmental conditions of controlled temperature, humidity, and gas mixture for the culture of cells. Preferred conditions are between about 34° C. to about 38° C., more preferably 37±1° C. with an atmosphere between about 5-10±1% CO₂ and a relative humidity (Rh) between about 80-90%.

Methods for providing epidermal cells to a dermal substrate, and methods for their culture, including induction of epidermal differentiation and cornification to form a differentiated keratinocyte layer are known in the art and are described in U.S. Pat. No. 5,712,163 to Parenteau, et al. and in U.S. Pat. No. 5,536,656 to Kemp, et al., in Wilkins (1994), supra, and in Parenteau (1994), supra, the teachings of which are incorporated herein by reference. Typically to perform the epidermalization of the cell-matrix construct, keratinocytes are seeded to the cell-matrix construct and cultured thereon until the layer is about one to three cell layers thick. The keratinocytes are then induced to differentiate to form a multilayer epidermis and are then induced to cornify to form a stratum corneum.

In the method of forming a differentiated epidermal layer, subcultured keratinocytes are taken from the cell stock and their cell numbers are expanded. When a necessary number of cells have been obtained, they are released from the culture substrate, suspended, counted, diluted and then seeded to the top surface of the cell-matrix construct at a density between about 4.5×10³ cells/cm² to about 5.0×10⁵ cells/cm², more preferably between about 1.0×10⁴ cells/cm² to about 1.0×10⁵ cells/cm², and most preferably at about 4.5×10⁴ cells/cm². The constructs are then incubated for between about 60 to about 90 minutes at 37±1° C., 10% CO₂ to allow the keratinocytes to attach. After the incubation, the constructs are submerged in epidermalization medium. After a sufficient length of time in culture, the keratinocytes proliferate and spread to form a confluent monolayer across the cell-matrix construct. Once confluent, the cell media formulation is changed to differentiation medium to induce cell differentiation. When a multilayer epithelium has formed, cornification media is then used and the culture is brought to the air-liquid interface. For the differentiation and cornification of keratinocytes, the cells are exposed to a dry or low humidity air-liquid interface. A dry or low-humidity interface can be characterized as trying to duplicate the low moisture levels of skin. With time, keratinocytes will express most or all keratins and other features found in native skin when exposed to these conditions.

When fully formed, the epidermal layer is a multilayered, stratified, and well-differentiated layer of keratinocytes that exhibit a basal layer, a suprabasal layer, a granular layer and a stratum corneum. Rudiments of basement membrane or a complete basement membrane are present at the dermal-epidermal junction and appears thickest around hemidesmosomes, marked by anchoring fibrils that are comprised of type VII collagen, as visualized by transmission electron microscopy (TEM). The anchoring fibrils are seen exiting from areas of basement membrane formation and entrapping the collagen fibrils in the dermal layer. These anchoring fibrits, as well as other basement membrane components, are secreted by keratinocytes. It is also known that while keratinocytes are capable of secreting basement membrane components on their own, a recognizable basement membrane will not form in the absence of fibroblasts. Immunohistochemical staining of the skin construct of the present invention has also shown that laminin, a basement membrane protein is present.

In their formation, the cultured skin constructs are nourished by contacting a culture medium that becomes conditioned by the cells in the skin construct as they metabolize components from the medium and secrete cytokines and other proteins into it. A defined medium means a culture medium for use in cell culture that contains chemically defined components and is free of undefined animal organ or tissue extracts, for example, serum, pituitary extract, hypothalamic extract, placental extract, or embryonic extract or proteins and factors secreted by feeder cells. In a most preferred embodiment, the media is free of undefined components and defined biological components derived from non-human sources. Although the addition of undefined components is not preferred, they may be used in accordance with the disclosed methods at any point in culture in order to fabricate successfully a tissue construct. When the invention is carried out utilizing screened human cells cultured using chemically defined components derived from no non-human derived biological components, the resultant tissue construct is a defined human tissue construct. The advantages in using such a construct to produce the conditioned medium of the invention is the elimination of the concern that adventitious animal or cross-species virus contamination and infection may be present in the tissue construct or the conditioned medium.

Culture medium, when fresh and unused, is comprised of a nutrient base usually further supplemented with other components. The skilled artisan can determine appropriate nutrient bases in the art of animal cell culture with reasonable expectations for successfully producing a tissue construct and the conditioned medium of the invention. Many commercially available nutrient sources are useful on the practice of the present invention. These include commercially available nutrient sources which supply inorganic salts, an energy source, amino acids, and B-vitamins such as Dulbecco's Modified Eagle's Medium (DMEM); Minimal Essential Medium (MEM); M199; RPMI 1640; Iscove's Modified Dulbecco's Medium (EDMEM). Minimal Essential Medium (MEM) and M199 require additional supplementation with phospholipid precursors and non-essential amino acids. Commercially available vitamin-rich mixtures that supply additional amino acids, nucleic acids, enzyme cofactors, phospholipid precursors, and inorganic salts include Ham's F-12, Ham's F-10, NCTC 109, and NCTC 135. Albeit in varying concentrations, all basal media provide a basic nutrient source for cells in the form of glucose, amino acids, vitamins, and inorganic ions, together with other basic media components. The most preferred base medium of the invention comprises a nutrient base of either calcium-free or low calcium Dulbecco's Modified Eagle's Medium (DMEM), containing glucose at 4.5 g/L, magnesium and L-glutamine at 7.25 mM, without sodium pyruvate, and Ham's F-12 in a 3-to-1 ratio.

The base medium is supplemented with components such as amino acids, growth factors, and hormones. Defined culture media for the culture of cells of the invention are described in U.S. Pat. No. 5,712,163 to Parenteau and in International PCT Publication No. WO 95/31473, the disclosures of which are incorporated herein by reference. Other media are known in the art such as those disclosed in Ham and McKeehan, Methods in Enzymology, 58:44-93 (1979), or for other appropriate chemically defined media, in Bottenstein et al., Methods in Enzymology, 58:94-109 (1979). In the preferred embodiment, the base medium is supplemented with the following components known to the skilled artisan in animal cell culture: insulin, transferrin, triiodothyronine (T3), and either or both ethanolamine and o-phosphoryl-ethanolamine, wherein concentrations and substitutions for the supplements may be determined by the skilled artisan.

Insulin is a polypeptide hormone that promotes the uptake of glucose and amino acids to provide long term benefits over multiple passages. Supplementation of insulin or insulin-like growth factor (IGF) is necessary for long term culture as there will be eventual depletion of the cells' ability to uptake glucose and amino acids and possible degradation of the cell phenotype. Insulin supplementation is advisable for serial cultivation and is provided to the media at a concentration range of preferably between about 0.5 μg/ml to about 50 μg/ml, more preferably at about 5 μg/ml. Appropriate concentrations for the supplementation of insulin-like growth factor, such as IGF-1 or IGF-2, may be easily determined by one of skill in the art for the cell types chosen for culture.

Transferrin is in the medium for iron transport regulation. Iron is an essential trace element found in serum. As iron can be toxic to cells in its free form, in serum it is supplied to cells bound to transferrin at a concentration range of preferably between about 0.05 to about 50 μg/ml, more preferably at about 5 μg/ml.

Triiodothyronine (T3) is a basic component and is the active form of thyroid hormone that is included in the medium to maintain rates of cell metabolism. Triiodothyronine is supplemented to the medium at a concentration range between about 0 to about 400 ρM, more preferably between about 2 to about 200 ρM and most preferably at about 20 ρM.

Either or both ethanolamine and o-phosphoryl-ethanolamine, which are phospholipids, are added whose function is an important precursor in the inositol pathway and fatty acid metabolism, Supplementation of lipids that are normally found in serum is necessary in a serum-free medium. Ethanolamine and o-phosphoryl-ethanolamine are provided to media at a concentration range between about 10⁻⁶ to about 10⁻² M, more preferably at about 1×10⁻⁴ M.

Throughout the culture duration, the base medium is additionally supplemented with other components to induce synthesis or differentiation or to improve cell growth such as hydrocortisone, selenium, and L-glutamine.

Hydrocortisone has been shown in keratinocyte culture to promote keratinocyte phenotype and therefore enhance differentiated characteristics such as involucrin and keratinocyte transglutaminase content (Rubin et al., J. Cell Physiol., 138:208-214 (1986)). Therefore, hydrocortisone is a desirable additive in instances where these characteristics are beneficial such as in the formation of keratinocyte sheet grafts or skin constructs. Hydrocortisone may be provided at a concentration range of about 0.04 μg/ml to about 4.0 μg/ml, most preferably at about 0.4 μg/ml.

Selenium is added to serum-free media to resupplement the trace elements of selenium normally provided by serum. Selenium may be provided at a concentration range of about 10⁻⁹ M to about 10⁻⁷ M; most preferably at about 5.3×10⁻⁸M.

The amino acid L-glutamine is present in some nutrient bases and may be added in cases where there is none or insufficient amounts present. L-glutamine may also be provided in stable form such as that sold under the mark, GlutaMAX-1™ (Gibco BRL, Grand Island, N.Y.). GlutaMAX-1™ is the stable dipeptide form of L-alanyl-L-glutamine and may be used interchangeably with L-glutamine and is provided in equimolar concentrations as a substitute to L-glutamine. The dipeptide provides stability to L-glutamine from degradation over time in storage and during incubation that can lead to uncertainty in the effective concentration of L-glutamine in medium. Typically, the base medium is supplemented with preferably between about 1 mM to about 6 mM, more preferably between about 2 mM to about 5 mM, and most preferably 4 mM L-glutamine or GlutaMAX-1™.

Growth factors such as epidermal growth factor (EGF) may also be added to the medium to aid in the establishment of the cultures through cell scale-up and seeding. EGF in native form or recombinant form may be used. Human forms, native or recombinant, of EGF are preferred for use in the medium when fabricating a skin equivalent containing no non-human biological components. EGF is an optional component and may be provided at a concentration between about 1 to about 15 ng/mL, more preferably between about 5 to about 10 ng/mL.

The medium described above is typically prepared as set forth below. However, it should be understood that the components of the present invention may be prepared and assembled using conventional methodology compatible with their physical properties. It is well known in the art to substitute certain components with an appropriate analogue or functionally equivalent acting agent for the purposes of availability or economy and arrive at a similar result. Naturally occurring growth factors may be substituted with recombinant or synthetic growth factors that have similar qualities and results when used in the performance of the invention.

Media in accordance with the present invention are sterile. Sterile components are bought or rendered sterile by conventional procedures, such as filtration, after preparation. Proper aseptic procedures were used throughout the following Examples. DMEM and F-12 are combined and the individual components are then added to complete the medium. Stock solutions of all components can be stored at −20° C., with the exception of nutrient source that can be stored at 4° C. All stock solutions are prepared at 500× final concentrations listed above. A stock solution of insulin, transferrin and triiodothyronine (all from Sigma) is prepared as follows: triiodothyronine is initially dissolved in absolute ethanol in 1N hydrochloric acid (HCl) at a 2:1 ratio. Insulin is dissolved in dilute HCl (approximately 0.1N) and transferrin is dissolved in water. The three are then mixed and diluted in water to a 500× concentration. Ethanolamine and o-phosphoryl-ethanolamine are dissolved in water to 500× concentration and are filter sterilized. Progesterone is dissolved in absolute ethanol and diluted with water. Hydrocortisone is dissolved in absolute ethanol and diluted in phosphate buffered saline (PBS). Selenium is dissolved in water to 500× concentration and filter sterilized. EGF is purchased sterile and is dissolved in PBS. Adenine is difficult to dissolve but may be dissolved by any number of methods known to those skilled in the art. Human serum albumin (HSA) or bovine serum albumin (BSA) may be added for prolonged storage to maintain the activity of the progesterone and EGF stock solutions. The medium can be either used immediately after preparation or, stored at 4° C. If stored, EGF should not be added until the time of use.

The mode of supplying fresh medium to cultures is done by pipetting, decanting, or pumping the medium into the culture apparatus. Conditioning of the medium occurs by contacting the medium with a cultured skin construct for a sufficient amount of time, usually for about 6 hours to 3 days or more to allow for the construct to absorb or take up nutrients and the like from the fresh medium and secrete cytokines into the medium. Since the cultured skin construct is in a constant metabolic state, only a short amount of time is needed to condition the medium. It is preferred that the construct and the medium contact each other for the exchange until the nutrients are nearly depleted from the fresh medium.

Conditioned medium is removed and collected from the cultures by pipetting, aspirating, decanting, draining, siphoning, or pumping at the time of each exchange of the conditioned medium with fresh medium. In the fabrication of a cultured skin equivalent, it is preferred that the conditioned medium be collected from the apparatus containing the constructs when both dermal fibroblasts and epidermal cells are present together in the construct. The conditioned media collections may be used individually as individual collections, or pooled together. The development of a cultured skin construct is marked with a number of events that produce a conditioned medium having a varying cytokine profile at each collection point. As separate collections, the conditioned medium will have certain cytokines that may be desirable for a particular treatment indication or product. When combined by pooling the collections together, the conditioned medium will have a broader range of cytokines for treatments or products.

Another mode of collection of cytokines of the invention is from the absorbent pad underlying the membrane on which the skin construct is formed. The pad is disposed beneath the membrane to wick medium to the membrane at airlift, when the culture is raised to the air-liquid interface to aid in cornification of the keratinocyte cell layer. The pad may be of any absorbent material but is preferably non-toxic and compatible with the cell cultures, such as cotton. Referring to FIG. 1, the pad is disposed along the bottom surface of membrane 24 between the membrane 24 on the bottom of outer chamber 60 The pad is shown to have higher concentrations of certain cytokines. While not wishing to be bound by theory, because the pad is in close opposition to the developing skin construct, it collects many of the cytokines secreted by the skin construct. The cytokines can be utilized white still in the pad when it is used as a bandage or part of a bandage or they can be extracted or drained from the pad.

Once collected, the conditioned medium is used as is collected or further processing is performed on the medium for purification or ease in application or storage before use. The conditioned medium may be lyophilized or evaporated to remove the liquid, or water, portion of the composition. Removal of water leaves a crystalline powder form of the conditioned medium containing the cultured skin agents: cytokines, proteins and extracellular matrix components, with decreased volume. This form makes it easier to prepare products containing higher dosages of the cultured skin agents composition without diluting the preparation and thus making it easier to store because of its decreased volume.

The conditioned medium may also be concentrated using a filtration method, particularly one with a molecular weight cut-off or a series of molecular weight filters. The use of molecular weight filters will remove large components found in medium such as albumin and those found in serum. Other filtration and dialysis methods may be used to remove salt from the cell product composition. The cultured skin agents may be further purified, fragmented, or conjugated to form a pure cytokine, protein, or extracellular matrix compositions or enhanced for directed delivery to a particular tissue, tissue structure or cell type.

The conditioned medium containing cytokines produced by skin constructs or the cytokines of the invention alone are useful in cell culture. The conditioned medium containing cytokines are used to grow and sustain cell lines by increasing cell-proliferation and generation of vital new skin cells, control the proliferation and differentiation of stem and progenitor cells, and mesenchymal differentiation (such as differentiation of mesenchymal cells to muscle cells). The conditioned medium is also used for making other tissue constructs for inhibiting or stimulating cell growth in particular layers or directions. The effect of the conditioned medium is concentration dependent, with higher concentrations producing a greater effect than lower concentrations.

The cultured skin agent compositions of the invention are particularly useful in preparations used in treating skin. Thus, a preferred embodiment of the invention comprises a conditioned cell culture medium containing any one or more of the following: cytokines, proteins, and extracellular matrix components, that are synthesized and secreted from cultured skin cells for use as a pharmaceutical preparation or a skincare product. In another preferred embodiment, the invention is a skin care composition comprising cultured skin agents synthesized and secreted from cultured skin cells and a carrier agent. The type of the compositions containing the cultured skin agents to be formulated will depend on the particular form of the agent and its intended use. Those of skill in treating epithelial tissues can determine the effective amount of cultured skin agents to be formulated in a pharmaceutical or cosmetic preparation. In a preferred embodiment, the invention is a cosmetic preparation, for topical administration to skin, containing conditioned medium components to care for and improve the skin's appearance. The cosmetic preparation may be used as or as an ingredient of the following non-limiting product examples: moisturizers, night creams, foundation creams, suntan lotions, sunscreens, hand lotions, make-up and make-up bases, masks, or ointments.

A particular benefit of the invention is a simple method of topical administration to the skin of a composition for increasing generation and proliferation of epidermal cells, decreasing epidermal cell senescence, or both, in a human. The method does not require the intact skin to have been pretreated to stimulate cell growth, making it a particularly simple method of topical administration to the skin not requiring abrading of the intact skin by a plastic surgery technique or wounding in any way. However, in one preferred embodiment of the invention, the skin is pretreated to remove all or some layers of the stratum corneum. The pretreatment can be mechanical, such as abrading, for example, with a particulate scrub, loofa, or the like or can be chemical, including biochemical, such as treatment with a keratolytic agent, such as alpha-hydroxy acid or retin-A, or with a cosmetically acceptable oil. Surgical abradement using mechanical, chemical or laser means, may also be performed.

The cultured skin agent formulations used in the method of the invention are most preferably applied in the form of appropriate compositions comprising the cultured skin agents from conditioned medium and a carrier agent. The carrier should be substantially inert so as not to react with the active component and diminish its activity. It is preferable that the carrier enhances and improves the permeation of the cytokines into the skin to increase their efficacy. Suitable inert carriers include water, alcohol polyethylene glycol, mineral oil or petroleum gel, propylene glycol and others known in the art.

To prepare the pharmaceutical compositions of this invention, an effective amount of the particular cultured skin agents as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for topical or percutaneous administration. Also included are solid form preparations that are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin.

In addition to the direct topical application of the cultured skin agent preparations, the compositions of this invention can be topically administered by other methods, for example, encapsulated in a temperature and/or pressure sensitive matrix or in film or solid carrier which is soluble in body fluids and the like for subsequent release, preferably sustained-release of the active component.

As appropriate compositions for topical application there may be cited all compositions usually employed for topically administering therapeutics, e.g., creams, jellies, dressings, shampoos, tinctures, pastes, ointments, salves, powders, emulsions, liquid or semi-liquid formulation and the like. Application of said compositions may be by aerosol, such as with a propellant such as air, nitrogen, carbon dioxide, a freon, or without a propellant such as a pump spray, atomizer, drops, lotions, or a semisolid such as a thickened composition which can be applied by a swab. In particular, semisolid compositions such as salves, creams, pastes, jellies, ointments and the like will conveniently be used.

The cultured skin agents of the present invention can be used, as stated above, for the many applications that can be considered skin care uses, such as to maintain skin with a youthful appearance. One way of retaining such appearance is to cease or reverse cellular senescence in skin cells. A large number of studies have shown that normal diploid cells undergo numerous cellular, physiological, biochemical and molecular changes during serial passaging in vitro. Most of these changes are progressive and accumulative and lead to an irreversible cessation of proliferation, followed by cell death. These changes have been considered as indicative of cellular aging in vitro. In short, in vivo and in vitro aging can be summarized as a failure to repair which lead to cell death. Similarly, these events occur in vivo, and are visually appreciated in skin. Many researchers are working to cease or reverse senescence to maintain populations young, healthy, synthetic and proliferative cells in patient tissues.

Skin care compositions known in the art for topical use on skin, preferably hypoallergenic and pH controlled are especially preferred, and include toilet waters, packs, lotions, skin milks or milky lotions. The preparations contain, besides the cultured skin agents, components usually employed in such preparations to function as carriers for the cultured cytokines. Examples of such carrier components are oils, fats, waxes, surfactants, humectants, thickening agents, antioxidants, viscosity stabilizers, chelating agents, buffers, preservatives, perfumes, dyestuffs, lower alkanols, and the like. If desired, further ingredients may be incorporated in the compositions, e.g. anti-inflammatory agents, antibacterials, antifungals, disinfectants, vitamins, sunscreens, antibiotics, or other anti-acne agents.

Examples of oils as a carrier agent comprises fats and oils such as olive oil and hydrogenated oils, waxes such as beeswax and lanolin; hydrocarbons such as liquid paraffin, ceresin, and squalene; fatty acids such as stearic acid and oleic acid; alcohols such as cetyl alcohol, stearyl alcohol lanolin alcohol, and hexadecanol; and esters such as isopropyl myristate, isopropyl palmitate and butyl stearate. As examples of surfactants as carrier agents, there may be cited anionic surfactants such as sodium stearate, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrocloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (such as the materials sold under the trademark “Pluronic”), polyoxyethylene castor oil, and polyoxyethylene lanolin. Examples of humectants as carrier agents include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants comprise butylated hydroxytoluene, butylated hydroxyanisole, propyl gallate, citric acid and ethoxyquin; examples of chelating agents include disodium edetate and ethanehydroxy diphosphate; examples of buffers as carrier agents comprise citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid.

For preparing ointments, creams, toilet waters, skin milks, and the like, to typically from about 0.01 to about 90% in particular from about 0.1 to about 20% and more in particular from about 0.2 to about 25% of the active ingredient, e.g., of the cultured cytokines, will be incorporated in the compositions. In ointments or creams, the carrier, for example, consists of 1 to 20%, in particular 5 to 15% of a humectant, 0.1 to 10% in particular from 0.5 to 5% of a thickener and water; or said carrier may consist of 70 to 99%, in particular 20 to 95% of a surfactant, and 0 to 20%, in particular 2.5 to 15% of a fat; or 80 to 99.9% in particular 90 to 99% of a thickener; or 5 to 15% of a surfactant, 2-15% of a humectant, 0 to 80% of an oil, very small (<2%) amounts of preservative, coloring agent and/or perfume, and water. In a toilet water, the carrier for example consists of 2 to 10% of a lower alcohol, 0.1 to 10% or in particular 0.5 to 1% of a surfactant, 1 to 20%, in particular 3 to 7% of a humectant, 0 to 5% of a buffer, water and small amounts (<2%) of preservative, dyestuff and/or perfume. In a skin milk, the carrier typically consists of 10-50% of oil, 1 to 10% of surfactant, 50-80% of water and 0 to 3% of preservative and/or perfume. In the aforementioned preparations, all % symbols refer to weight by weight percentage.

Particular compositions for use in the method of the present invention are those wherein the cultured skin agents are formulated in liposome-containing compositions that are functional carrier agents for the cultured skin agents. Liposomes are artificial vesicles formed by amphiphatic molecules such as polar lipids, for example, phosphatidyl cholines, ethanolamines and serines, sphingomyelins, cardiolipins, plasmalogens, phosphatidic acids and cerebiosides. Liposomes are formed when suitable amphiphatic molecules are allowed to swell in water or aqueous solutions to form liquid crystals usually of multilayer structure comprised of many bilayers separated from each other by aqueous material (also referred to as coarse liposomes). Another type of liposome known to be consisting of a single bilayer encapsulating aqueous material is referred to as a unilamellar vesicle. If water-soluble materials are included in the aqueous phase during the swelling of the lipids they become entrapped in the aqueous layer between the lipid bilayers.

Water-soluble active ingredients such as, for example, various salt forms of cultured skin agents, are encapsulated in the aqueous spaces between the molecular layers. Lipid soluble active ingredients of cultured cytokines, such as an organic mimetic, is predominantly incorporated into the lipid layers, although polar head groups may protrude from the layer into the aqueous space. The encapsulation of these compounds can be achieved by a number of methods. The method most commonly used involves casting a thin film of phospholipid onto the walls of a flask by evaporation from an organic solvent. When this film is dispersed in a suitable aqueous medium, multilamellar liposomes are formed. Upon suitable sonication, the coarse liposomes form smaller similarly closed vesicles.

Water-soluble active ingredients are usually incorporated by dispersing the cast film with an aqueous solution of the compound. The unencapsulated compound is then removed by centrifugation, chromatography, dialysis or other art-known suitable procedures. The lipid-soluble active ingredient is usually incorporated by dissolving it in the organic solvent with the phospholipid prior to casting the film. If the solubility of the material in the lipid phase is not exceeded or the amount present is not in excess of that which can be bound to the lipid, liposomes prepared by the above method usually contain most of the material bound in the lipid bilayers; separation of the liposomes from unencapsulated material is not required.

A particularly convenient method for preparing liposome formulated forms of therapeutics containing cultured skin agents is the method described in EP-A-253,619, incorporated herein by reference. In this method, single bilayered liposomes containing encapsulated cultured skin agents are prepared by dissolving the lipid component in an organic medium, injecting the organic solution of the lipid component under pressure into an aqueous component while simultaneously mixing the organic and aqueous components with a high speed homogenizer or mixing means, whereupon the liposomes are formed spontaneously.

The single bilayered liposomes containing the encapsulated cultured skin agents can be employed directly or they can be employed in a suitable pharmaceutically acceptable carrier for topical administration. The viscosity of the liposomes can be increased by the addition of one or more suitable thickening agents such as, for example xanthan gum, hydroxypropyl cellulose, hydroxypropyl methylcellulose and mixtures thereof. The aqueous component may consist of water alone or it may contain electrolytes, buffered systems and other ingredients, such as, for example, preservatives. Suitable electrolytes that can be employed include metal salts such as alkali metal and alkaline earth metal salts. The preferred metal salts are calcium chloride, sodium chloride and potassium chloride. The concentration of the electrolyte may vary from zero to 260 mM, preferably from 5 mM to 160 mM. The aqueous component is placed in a suitable vessel which can be adapted to effect homogenization by effecting great turbulence during the injection of the organic component. Homogenization of the two components can be accomplished within the vessel, or, alternatively, the aqueous and organic components may be injected separately into a mixing means located outside the vessel. In the latter case, the liposomes are formed in the mixing means and then transferred to another vessel for collection purpose.

The organic carrier component consists of a suitable non-toxic, pharmaceutically acceptable solvent such as, for example ethanol glycerol, propylene glycol and polyethylene glycol, and a suitable phospholipid that is soluble in the solvent. Suitable phospholipids that can be employed include lecithin, phosphatidycholine, phosphatidylserine, phosphatidylethanol amine, phosphatidylinositol, lysophosphatidylcholine and phospha-tidyl glycerol, for example. Other lipophilic additives may be employed in order to selectively modify the characteristics of the liposomes. Examples of such other additives include stearylamine, phosphatidic acid, tocopherol, cholesterol and lanolin extracts.

In addition, other ingredients that can prevent oxidation of the phospholipids may be added to the organic component. Examples of such other ingredients include tocopherol, butylated hydroxyanisole, butylated hydroxytoluene, ascorbyl palmitate and ascorbyl oleate. Preservatives such a benzoic acid, methyl paraben and propyl paraben may also be added.

Apart from the above-described compositions, use may be made of covers, e.g. plasters, bandages, dressings, gauze pads and the like, containing the composition of this invention with an appropriate amount of cultured skin agents. In some cases use may be made of plasters, bandages, dressings, gauze pads and the like which have been impregnated with a topical formulation containing the therapeutic formulation. Tissue sealants such as surgical glues to aid in wound closure may also contain cultured skin agents. A preferred example of a tissue sealant is fibrin glue due to its biocompatibility with cells. The sealants or in liquid form, appropriate for the addition and mixing in of the cultured skin agent composition. When the cultured skin agents of the invention are added to the sealants and the composition is applied to a wound to assist in wound closure, the cytokines enhance wound healing by the cells in the area the sealant is applied.

In a preferred method for treating skin with a cultured skin agent composition, the skin has first undergone a resurfacing treatment. All skin resurfacing treatments work essentially the same way. First, the outer layers of damaged skin are stripped away. Then, as new cells multiply and migrate into the resurfaced area during the healing process, a smoother, tighter, younger-looking skin surface appears. During the healing process, cultured skin agent compositions derived from conditioned medium are applied to the treated area to enhance and accelerate skin healing and regimentation. For superficial or medium resurfacing, the layers of skin tissue removed can be limited to the epidermis and papillary dermis. For deeper resurfacing, the upper levels of the reticular dermis can also be removed. Varied penetration allows treatment of specific spots or wrinkles.

In laser resurfacing, sometimes called “laser peel”, a carbon dioxide (CO₂) laser is used to remove areas of damaged or wrinkled skin, layer by layer. Laser resurfacing is performed using a beam of laser energy that vaporizes the upper layers of damaged skin at specific and controlled levels of penetration. The procedure is most commonly used to minimize the appearance of fine lines, especially around the mouth and the eyes; however, it is also effective in treating facial scars or areas of uneven pigmentation. Laser resurfacing may be performed on the whole face or in specific regions. Often, the procedure is done in conjunction with another cosmetic operation, such as a facelift or eyelid surgery.

“Dermabrasion” and “dermaplaning” help to refinish the skin's top layers through a method of controlled surgical scraping. The treatments soften the sharp edges of surface irregularities, giving the skin a smoother appearance. Dermabrasion is most often used to improve the look of facial skin left scarred by accidents or previous surgery, or to smooth out fine facial wrinkles, such as those around the mouth, but is also sometimes used to remove the pre-cancerous growths called keratoses. In dermabrasion, the surgeon scrapes away the outermost layer of skin with a rough wire brush, or a burr containing diamond particles, attached to a motorized handle. The scraping continues until the surgeon reaches the safest level that will make the scar or wrinkle less visible. Dermaplaning is commonly used to treat deep acne scars. In dermaplaning, the surgeon uses a hand-held instrument called a dermatome. Resembling an electric razor, the dermatome has an oscillating blade that moves back and forth to evenly skim off the surface layers of skin that surround the craters or other facial defects. This skimming continues until the lowest point of the acne scar or wrinkle becomes more even with the surrounding skin. Both dermabrasion and dermaplaning can be performed on small areas of skin or on the entire face. They can be used alone, or in conjunction with other procedures such as facelift, scar removal or revision, or chemical peel.

Chemical peels use a chemical solution to improve and smooth the texture of the facial skin by removing its damaged outer layers. Phenol, trichloroacetic acid (TCA), and alphahydroxy acids (AHAs) are used for this purpose. Although chemical peel may be performed in conjunction with a facelift, it is not a substitute for such surgery, nor will it prevent or slow the aging process. Alphahydroxy acids (AHAs), such as glycolic, lactic, or fruit acids are the mildest of the peel formulas and produce light peels. AHA peels may be used to treat fine wrinkling, areas of dryness, uneven pigmentation and acne. Various concentrations of an AHA may be applied weekly or at longer intervals to obtain the best result. An alphahydroxy acid, such as glycolic acid, can also be mixed with a facial wash or cream in lesser concentrations, containing the cytokines as part of a daily skin-care regimen to improve the skin's texture. Trichloroacetic acid (TCA) can be used in many concentrations, but it is most commonly used for medium-depth peeling. Fine surface wrinkles, superficial blemishes and pigment problems are commonly treated with one or more TCA treatments. Phenol is the strongest of the chemical solutions and produces a deep peel and sometimes lightens treated areas and affects skin pigmentation for the immediate term. It is used mainly to treat patients with coarse facial wrinkles, areas of blotchy or damaged skin caused by sun exposure, or pre-cancerous growths.

After a skin resurfacing procedure, skin is quite red and swollen, associated with some tingling, burning, or aching; any pain can be controlled with medications. Swelling subsides in a few days to a week and a scab or crust will form over the treated area as it begins to heal. This will fall off as a new layer of tight, pink skin forms underneath. When the procedure is over, the surgeon may choose to treat the resurfaced skin with applications of protective creams or ointments containing the cultured skin agent composition until healing is complete. If ointment is applied immediately after surgery, little or no scab will form. Some surgeons may also choose to apply a bandage over the treated areas that will cover and protect the healing skin for the first five to ten days. The ointment containing the cultured skin agent composition that is applied to the resurfaced area benefits the patient by providing growth factors that support the growth of young skin cells for faster healing and an improved cosmetic effect.

The following examples are provided to better explain the practice of the present invention and should not be interpreted in any way to limit the scope of the present invention. Those skilled in the art will recognize that various modifications can be made to the methods described herein while not departing from the spirit and scope of the present invention.

### EXAMPLES

### Example 1

### Culturing a Bilayer Skin Construct to Produce Conditioned Medium

Human neonatal foreskin fibroblasts (originated at Organogenesis, Inc. Canton, Mass.) were seeded at 5×10⁵ cells/162 cm² tissue culture treated flask (Costar Corp., Cambridge, Mass., cat #3150) and grown in growth medium. The growth medium consisted of: Dulbecco's Modified Eagle's medium (DMEM) (high glucose formulation, without L-glutamine, BioWhittaker, Walkersville, Md.) supplemented with 10% newborn calf serum (NBCS) (HyClone Laboratories, Inc., Logan, Utah) and 4 mM L-glutamine (BioWhittaker, Walkersville, Md.). The cells were maintained in an incubator at 37±1° C. with an atmosphere of 10±1% CO₂. The medium was replaced with freshly prepared medium every two to three days. After 8 days in culture, the cells had grown to confluence, that is, the cells had formed a packed monolayer along the bottom of the tissue culture flask, and the medium was aspirated from the culture flask. To rinse the monolayer, sterile-filtered phosphate buffered saline was added to the bottom of each culture flask and then aspirated from the flasks. Cells were released from the flask by adding 5 mL trypsin-versene glutamine (BioWhittaker, Walkersville, Md.) to each flask and gently rocking to ensure complete coverage of the monolayer. Cultures were returned to the incubator. As soon as the cells were released 5 ml of SBTI (Soybean Trypsin Inhibitor) was added to each flask and mixed with the suspension to stop the action of the trypsin-versene. The cell suspension was removed from the flasks and evenly divided between sterile, conical centrifuge tubes. Cells were collected by centrifugation at approximately 800-1000×g for 5 minutes.

An apparatus similar to that shown in FIG. 1—was used in conducting the work described hereinafter. The cover is removed for conducting operation but is otherwise kept in place to maintain sterility. Pertinent information regarding the apparatus is listed: Outer container 10 has a diameter of 38 mm and a capacity of 35 ml, The inner container 20 has a diameter of 24 mm and a capacity of 4 ml. The permeable member 24 consists of a polycarbonate membrane with a pore size of about 3 μm (micron) and a thickness of 5 μm (micron).

An acellular, hydrated collagen gel 25 was formed on the permeable member 24 as follows: A “premix”⁷ solution of 16.2 ml 10× Minimum Essential Medium (MEM), 1.6 ml 200 mM L-glutamine, 0.2 ml 50 mg/ml gentamycin, 18.0 ml fetal bovine serum, 5.0 ml 71.2 mg/ml sodium bicarbonate. The stock solutions were aseptically combined in the above sequence, and stored at 4° C. for approximately 30 minutes in a sterile 50 ml tube. About 27.8 g of 1 mg/ml collagen solution (extracted by acid from calf common digital extensor tendon) in 0.05% v/v acetic acid, was weighed out into a 50 ml tube and stored 4° C. for 30 minutes. About 8.2 ml of the pre-mix described above and 4 ml of DMEM complete (containing 10% FBS, 4 mM L-glutamine, 50 μg/ml gentamycin) was added and 1 ml aliquots were pipetted onto the membrane of the inner container 20 and allowed to gel at room temperature.

The dermal layer, a hydrated collagen gel containing cells, was cast with human dermal fibroblasts and seeded with human epidermal (epithelial) cells as described below. A general description of procedures and reagents may also be found in U.S. Pat. No. 4,485,096 to Bell, U.S. Pat. No. 5,536,656 to Kemp, et al., and U.S. Pat. No. 5,712,163 to Parenteau. The casting mixture for preparing the dermal layer included about 8.2 ml of the pre-mix described above to which was added to 27.8 g of a 1 mg/ml collagen solution in 0.05% v/v acetic acid, also described above, and 4 ml of human dermal fibroblasts at a density of 2.5×10⁵ cells/ml. Aliquots of about 3 ml were pipetted into the container 20 over the acellular, hydrated collagen gel 25 formed above and allowed to gel. About 4.5 ml Dulbecco's Minimum Essential Medium (DMEM) complete was added to the outside container 20 and then incubated at 36° C./10% CO₂ for 4 to 8 days to allow the cells to contract the collagen to form a contracted collagen lattice to serve as a dermal layer 52.

The following medium was prepared for providing epidermal cells to top surface of the dermal layer 52, a process referred to as epidermalization. Monolayer cultures of epidermal cells were cultured and harvested in a similar fashion to dermal fibroblasts, above. The epidermalization medium formulation consisted of a base mixture of Calcium Free DMEM and Ham's F-12 mixed at a volume per volume ratio of 3:1 was added the following components: 1.1 mM hydrocortisone, 5 μg/ml insulin, 5 μg/ml transferrin, 20 pM triiodothyronine (T3), 1×10⁻⁴ M ethanolamine, 1×10⁻⁴ M o-phosphorylethanolamine, 0.18 mM adenine, 2×10⁻⁹ M progesterone, 5.26×10⁻⁸ M selenium, 0.3% newborn calf serum, 10 ng/ml epidermal growth factor (EGF).

Epidermalization was initiated at 6 days after casting the tissue equivalent. The medium bathing the dermal construct above was removed from both the inside 20 and outside 10 containers. A 50 μl suspension of human epidermal cells (approximately 3.33×10⁶ cells/ml) was placed on the dermal construct. The container was then incubated at 36° C. and 10% CO₂ for 4 hours after which time 12.0 ml of epidermalization medium was then added to the outside chamber and 4 ml to the well. The cultures were then returned to the same incubator.

At two days post-epidermalization, differentiation of the epidermal layer was induced by adding calcium to the epidermalization medium formulation. The conditioned epidermalization medium was removed from the culture dish, set aside, and replaced with differentiation medium. Differentiation medium consisted of a base mixture of Calcium Free DMEM and Ham's F-12 mixed at a volume per volume ratio of 3:1 was added the following components: 1.1 mM hydrocortisone, 5 μg/ml insulin, 5 μg/ml transferrin, 20 pM triiodothyronine (T3), 1×10⁴ M ethanolamine, 1×10⁻⁴ M o-phosphorylethanolamine, 0.18 M adenine, 2×10⁻⁹ M progesterone, 5.26×10⁻⁸ M selenium, 0.3% newborn calf serum, 10 ng/ml epidermal growth factor (EGF) and 1.8 mM calcium chloride. The cultures were then returned to the same incubator.

At 5 days post epidermalization, the culture was airlifted to bring the surface of the forming epidermal layer of the cultured skin construct to the air-liquid interface, that is, to contact the epidermal surface to air. The conditioned differentiation medium was removed from both inside and outside chambers of the dish, set aside, and the inner container was removed and cotton pads were positioned in the interior of the bottom of the outer chamber 10 and cornification medium was added to the lower chamber to soak the pads. The inner container 20 was returned to rest on the soaked cotton pads with care taken to ensure that no air bubbles were trapped between the container and the pads. Cornification medium consisted of a base mixture of Calcium Free DMEM and Ham's F-12 mixed at a volume per volume ratio of 1:1 was added the following components: 1.1 mM hydrocortisone, 5 μg/ml insulin, 5 μg/ml transferrin, 20 pM triiodothyronine (T3), 1×10⁻⁴ M ethanolamine, 1×10⁻⁴ M o-phosphorylethanolamine, 0.18 M adenine, 5.26×10⁻⁸ M selenium, 2.0% newborn calf serum, and 2 mM sodium ascorbate. The cultured skin constructs were returned to the incubator and cultured at 35.5° C. and 10% CO₂.

Every 4 days the conditioned medium was removed, set aside, and replaced with fresh maintenance medium plus calcium. Maintenance medium consisted of a base mixture of Calcium Free DMEM and Ham's F-12 mixed at a volume per volume ratio of 1:1 was added the following components: 1.1 mM hydrocortisone, 5 μg/ml insulin, 5 μg/ml transferrin, 20 pM triiodothyronine (T3), 1×10⁻⁴ M ethanolamine, 1×10⁻⁴ M o-phosphorylethanolamine, 0.18 mM adenine, 5.26×10⁻⁸ M selenium, and 1.0% newborn calf serum. At this point, a well stratified epidermal layer 28 had formed on the top surface of the dermal layer 52 that exhibited many of the morphological and biochemical features of normal native skin.

The conditioned epidermalization, differentiation, cornification, and maintenance media collected from the process of fabricating a cultured skin construct were tested using cell proliferation, migration, and ELISA assays in either originally collected.

### Example 2

### In Vitro Formation of a Skin Construct Formed from Endogenously Produced Collagenous Matrix by Human Neonatal Foreskin Fibroblasts

Conditioned medium was produced by bilayer skin constructs having a matrix endogenously produced by dermal fibroblasts as described in International PCT Patent Application Publication No. WO 00/29553 to Murphy, the disclosure of which is incorporated herein.

Human neonatal foreskin fibroblasts were cultured, expanded in number, released from their substrate, counted, concentrated, and then resuspended to a concentration of 3×10⁶ cells/ml, and seeded on to 0.4 micron pore size, 24 mm diameter tissue culture treated membrane inserts in a six-well tray at a density of 3.0×10⁶ cells/TW (6.6×10⁵ cells/cm²). These cells were then maintained with media exchanges every two to three days with fresh media for 25 days. More specifically the medium contained: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 0.4 μg/ml hydrocortisone (Sigma, St. Louis, Mo.), 1×10⁻⁴M ethanolamine (Fluka, Ronkonkoma, N.Y. cat. #02400 ACS grade), 1×10⁻⁴ M o-phosphoryl-ethanolamine (Sigma, St. Louis, Mo.), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo.).

Using a 25-day dermal constructs as formed above, normal human neonatal foreskin epidermal keratinocytes were seeded on the top surface of the cell-matrix construct to form the epidermal layer of the skin construct. The medium was aseptically removed from the culture insert and its surrounds Normal human epidermal keratinocytes that had been scaled up to passage 4 from frozen subculture cell stock to confluence were used. Cells were then released from the culture dishes using trypsin-versene, pooled, centrifuged to form a cell pellet, resuspended in epidermalization medium, counted and seeded on top of the membrane at a density of 4.5×10⁴ cells/cm². The constructs were then incubated for 90 minutes at 37±1° C., 10% CO₂ to allow the keratinocytes to attach. After the incubation, the constructs were submerged in epidermalization medium. The epidermalization medium is composed of: a 3:1 base mixture of Dulbecco's Modified Eagle's Medium (DMEM) (containing no glucose and no calcium, BioWhittaker, Walkersville, Md.) and Hams F-12 medium (Quality Biologics Gaithersburg, Md.), supplemented with 0.4 μg/ml hydrocortisone (Sigma St. Louis, Mo.), 1×10⁻⁴ M ethanolamine (Fluka, Ronkonkoma, N.Y.), 1×10⁻⁴ M o-phosphoryl-ethanolamine (Sigma, St. Louis, Mo.), 5 μg/ml insulin (Sigma, St. Louis, Mo.), 5 μg/ml transferrin (Sigma, St. Louis, Mo.), 20 ρM triiodothyronine (Sigma, St. Louis, Mo.), 6.78 ng/ml selenium (Aldrich), 24.4 μg/ml adenine (Sigma Aldrich Fine Chemicals Company, Milwaukee, Wis.), 4 mM L-glutamine (BioWhittaker, Walkersville, Md.), 50 μg/ml L-ascorbate sodium salt (Sigma Aldrich Fine Chemicals Company, Milwaukee, Wis.), 16 μM linoleic acid (Sigma, St. Louis, Mo.), 1 μM tocopherol Acetate (Sigma, St. Louis, Mo.) and 50 μg/ml gentamicin sulfate (Amersham, Arlington Heights, Ill.). The constructs were cultured in the epidermalization medium for 2 days at 37±1° C., 10±1% CO₂.

After 2 days the medium was exchanged with fresh medium composed as above, and returned to the incubator set at 37±1° C., 10±1% CO₂ for 2 days. After the 2 days, the carrier containing the construct was aseptically transferred to new culturing trays with sufficient media to achieve a fluid level just to the surface of the carrier membrane to maintain the developing construct at the air-liquid interface. The air contacting the top surface of the forming epidermal layer allows stratification of the epithelial layer. The constructs were incubated at 37±1° C., 10% CO₂, and low humidity, in media with media changes every 2-3 days for 7 days. This medium contained a 1:1 mixture of Dulbecco's modified Eagle's medium (DMEM) (containing no glucose and no calcium, BioWhittaker, Walkersville, Md.), Hams F-12 medium (Quality Biologics, Gaithersburg, Md.), supplemented with 0.4 μg/ml hydrocortisone (Sigma, St. Louis, Mo.), 5×10⁻⁴ M ethanolamine (Fluka, Ronkonkoma, N.Y.), 5×10⁻⁴ M o-phosphoryl-ethanolamine (Sigma, St. Louis, Mo.), 5 μg/ml insulin (Sigma, St. Louis, Mo.), 5 μg/ml transferrin (Sigma, St. Louis, Mo.), 20 ρM triiodothyronine (Sigma, St. Louis, Mo.), 6.78 ng/ml selenium (Sigma Aldrich Fine Chemicals Company), 24.4 μg/ml adenine (Sigma Aldrich Fine Chemicals Company), 4 mM L-glutamine (BioWhittaker, Walkersville, Md.), 2.65 μg/ml calcium chloride (Mallinekrodt, Chesterfield, Mo.), 16 μM linoleic acid (Sigma, St. Louis, Mo.), 1 μM tocopherol acetate (Sigma, St. Louis, Mo.), 1.25 mM serine (Sigma, St. Louis, Mo.), 0.64 mM choline chloride (Sigma, St. Louis, Mo.) and 50 μg/ml gentamicin sulfate (Amersham, Arlington Heights, Ill.). The cultures were fed every 2-3 days, for 14 days.

After the application of epidermal cells to the dermal construct, conditioned media are aspirated from the culture tray containing the developing skin construct and frozen until use as is or treated to concentrate or purify the cell-produced skin agents.

### Example 3

### Administering a Composition Containing Cultured Skin Agents to an Individual

To determine the effects of a topical cream containing the conditioned media of Example 1 on senescence of the skin, subjects are enrolled in a study to compare the test composition to a control composition not containing cultured skin agents from conditioned tissue culture media. These volunteer subjects are treated topically with two different cream preparations. The test areas are divided into four regions on each forearm two centimeters distal to the antecubital fossa and each arm two centimeters proximal to the antecubital fossa.

Each test area is treated twice daily for 60 days. One milliliter of the respective cream is applied to each test area during the dosing. At the end of the 60-day period, respective photographs are obtained from each test site on each subject; in addition, 2 mm punch biopsies are obtained from each test area. These biopsies are incubated for twelve hours in a trypsin solution to separate epidermis from dermis. Once the epidermis was separated it is submitted for flow cytometric analysis to determine the percentage of keratinocytes in the S-Phase.

Results demonstrate that the test preparation increases the cellular division rates significantly over controls suggesting that the cultured skin agents from conditioned tissue culture media exerts a mitogenic effect that has a role in reversing or ceasing the senescent epidermal cell cycle.

To determine if a dermal affect is produced by the strong mesodermal effects of the conditioned media composition, the dermis is further analyzed for hydroxyproline content as an indirect measure of cellular activity. The data demonstrates that by hydroxyproline assay the control preparation seems to exert no statistical effect on the dermis whereas the test cream containing the cultured skin agent preparation obtained from cultured skin constructs produces an increase in the hydroxyproline content.

### Example 4

### Clonal Density Culture of Keratinocytes: Evaluation of Colony Size

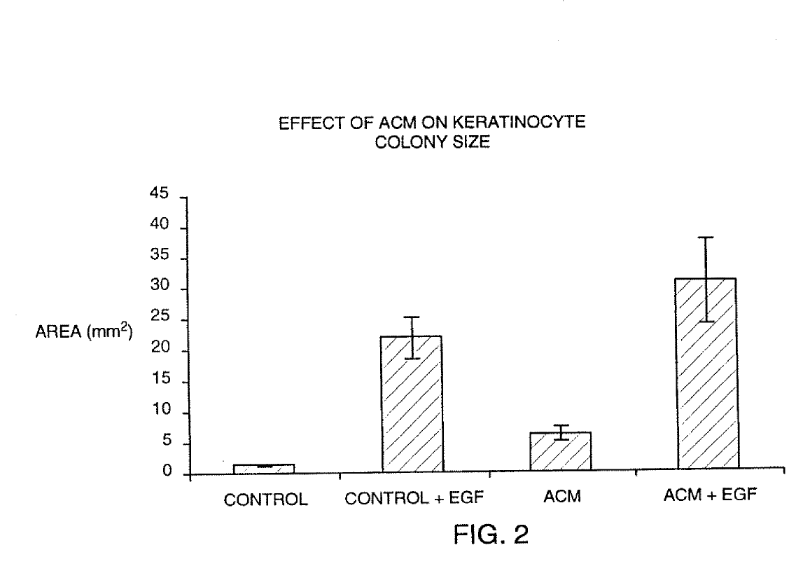

The effect of conditioned medium from the culture of cultured bilayer skin constructs was evaluated on keratinocyte migration using the method taught in Green H, Kehinde O, Thomas J: “Growth of human epidermal cells into multiple epithelia suitable for grafting.” Proceedings of the National Academy of Science USA, 76:5665-5668 (1979), the teachings of which are incorporated herein by reference. Conditioned maintenance medium was removed from cultured skin constructs between 10 and 12 days post-air-lift (PAL). Control media were fresh, unconditioned maintenance medium mixed 1:1 with fresh FAD medium and fresh FAD medium (100%); test medium was conditioned medium mixed 1:1 with fresh FAD medium. 100 mm or 60 mm Petri dishes were coated with type I collagen. Seeded to the collagen-coated dishes were 1.5-5.0×10⁵ Mitomycin C treated 3T3 cells used as a feeder layer. The 3T3 cells were culture in FAD medium 10% FCS without EGF. Keratinocytes were seeded at 100 cells per 100 mm dish or 50 cells per 60 mm dish. Medium changes were done every 2-3 days and the cultures were fixed at day 12 of culture. Cells were visualized on the dishes using Acid Fucsin staining and cell counts and area measures of the keratinocyte cell colonies were determined. Data are presented in Table 1 and in FIG. 2.

TABLE 1
Effect of Conditioned Medium on Keratinocyte Migration

| | Average Colony Size | |
|---|---|---|
| | (mm²) | Number of Colonies |
| 100% FAD | 1.2 | 121 |
| 50% unconditioned | 1.4 | 117 |
| maintenance medium/50% | | |
| FAD | | |
| 50% conditioned | 6.2 | 147 |
| maintenance medium/50% | | |
| FAD | | |

The results demonstrate a large effect of conditioned medium on keratinocyte colony size and number indicating that the conditioned medium contains bioactive components that increase keratinocyte migration over fresh control media.

### Example 5

### Measure of Keratinocyte Proliferation

To study the effects of conditioned medium from cultured skin constructs on keratinocyte proliferation, the 3T3 culture system described in Example 1 was used. The proliferation assay was performed in 24-well plates provided with a collagen coating. 3T3 feeder cells were seeded to the plates in FAD medium. Keratinocytes were seeded to the feeder layers at 1×10³ cells/well and cultured for 9 days with media changes every 2-3 days. Media conditions tested were:
A. 100% unconditioned maintenance medium.
B. 100% unconditioned maintenance medium+10 ng/ml EGF
C. 90% unconditioned maintenance medium 10% conditioned maintenance medium from cultured skin constructs between 10 and 12 days PAL.
D. 50% unconditioned maintenance medium 50% conditioned maintenance medium from cultured skin constructs between 10 and 12 days PAL.
E. 100% conditioned maintenance medium from cultured skin constructs between 10 and 12 days PAL.

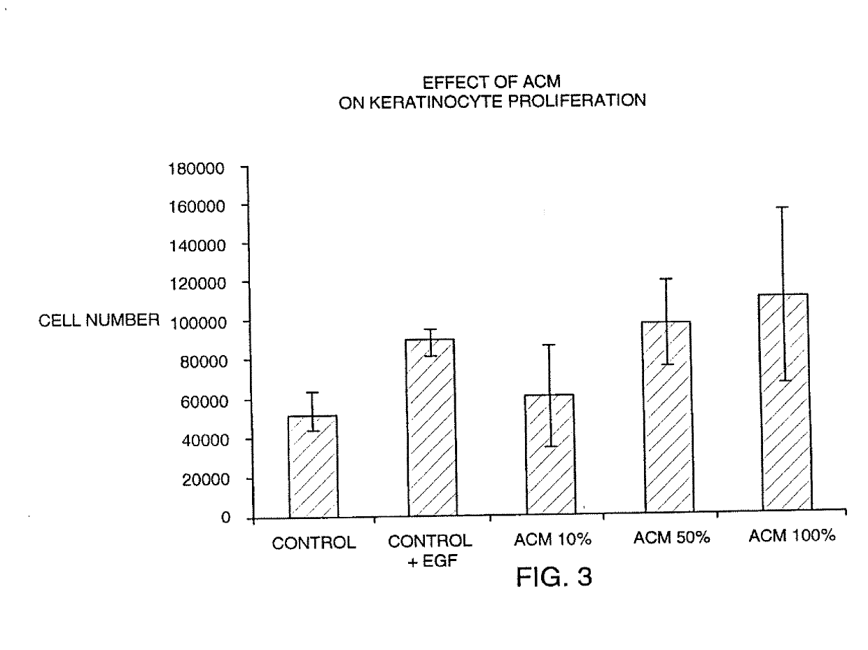

Results from the proliferation assay showed, as demonstrated in FIG. 3, that the medium of Condition E had increased proliferation over the unconditioned medium containing EGF of Condition B. Further, the 1:1 mix of unconditioned and conditioned media of Condition D had increased proliferation of keratinocytes over the 9:1 mix of unconditioned and conditioned media, respectively, of Condition C which, in turn, had a greater proliferative effect over the 100% unconditioned medium of Condition A. These results suggest that the conditioned medium contains other cytokines other than EGF that promote keratinocyte proliferation.

### Example 6

### Effect of Conditioned Medium on Cell Migration on a Fibrin Substrate

Cell migration assays were performed using a method for evaluating keratinocytes migration of Ronfard, V. and Barrandon, Y. as disclosed in International PCT Application Number WO 97/25617, the methods of which are incorporated herein by reference. A fibrin gel substrate was prepared on the bottoms of each dish according to the method.

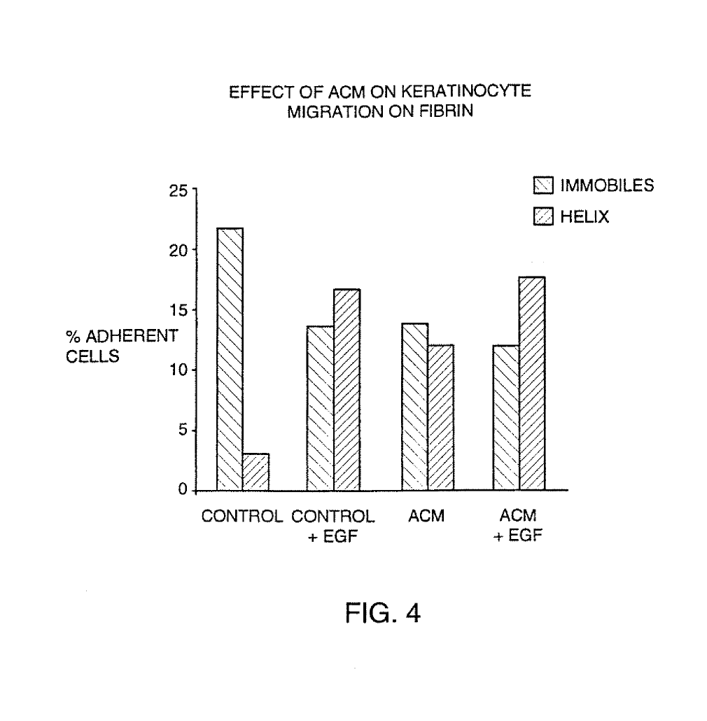

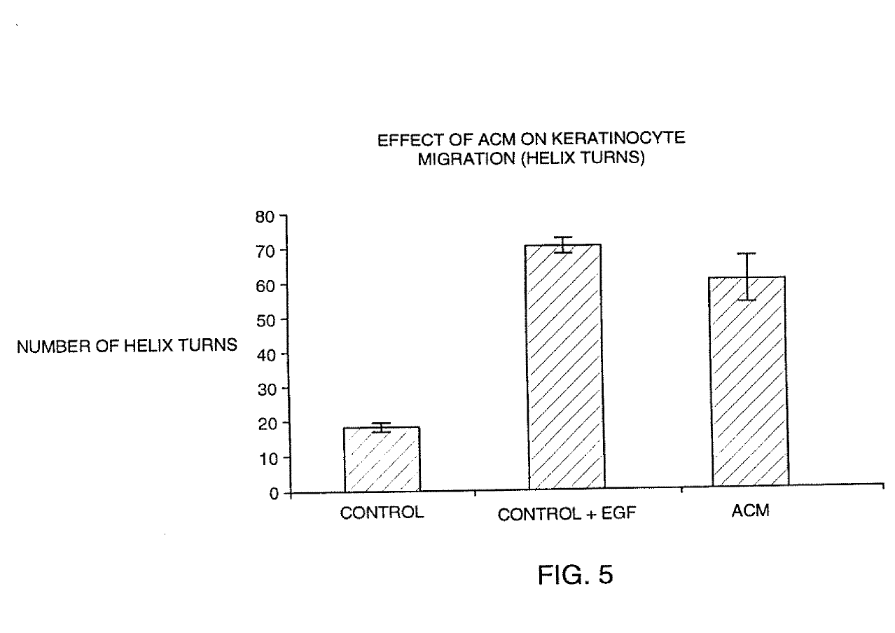

To the top of the fibrin substrate, 1×10⁴ keratinocytes were plated in 50% DMEM+10% fetal calf serum/50% test medium. Test media tested were: control medium without EGF, control medium containing EGF, and conditioned medium. The cultures were incubated at 37° C. for 20-24 hours; fixed, and the migrating cells were counted along with the helical turns made by the cells as they migrated into the fibrin gel substrate. Cell migration data are presented in FIGS. 4 and 5. FIG. 4 shows the number of adherent cells, both immobile cells and mobile cells that migrate in a helical pattern on the fibrin substrate. FIG. 5 shows the average number of helical turns the mobile cells make on the fibrin substrate. Cells in conditioned medium (ACM) make nearly as many turns as fresh control medium containing EGF indicating that there is a growth factor effect on inducing cell mobility suggesting that the conditioned medium (ACM) also contains growth factors.

### Example 7

### Cell Proliferation of Other Cells

Cell proliferation assays for endothelial cells, smooth muscle cells, and dermal fibroblasts were performed using the method described in Kratz and, Haegerstrand: “Conditioned Medium from Cultured Human Keratinocytes Has Growth Stimulatory Properties on Different Human Cell Types. Journal of Investigative Dermatology, 97:1039-1043 (1991), the teachings of which are incorporated herein by reference.

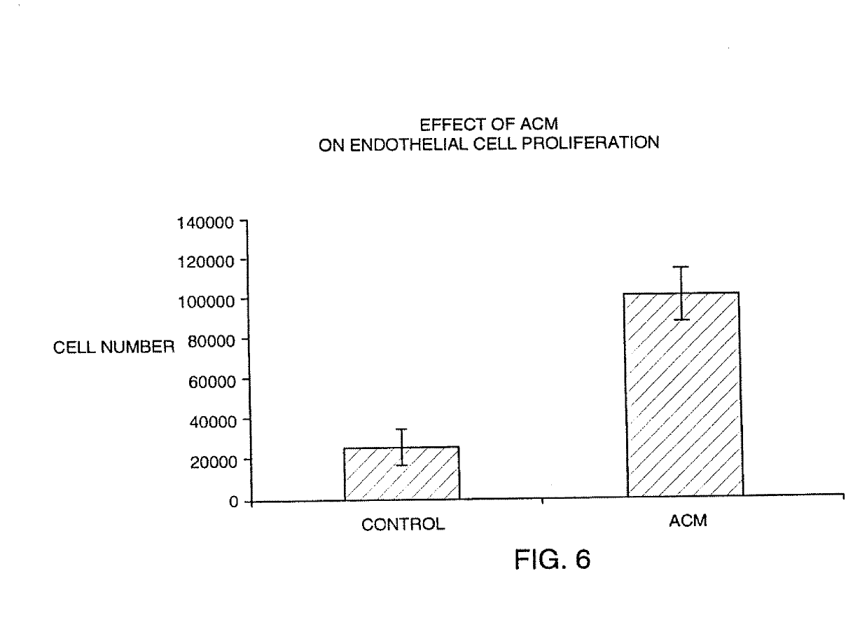

Endothelial cells, when cultured with condition medium taken from cultured skin constructs, exhibit enhanced proliferative activity over those cultured in control medium. Data for endothelial cell proliferation are presented in FIG. 6.

The proliferation activity of smooth muscle cells and dermal fibroblasts (separately) were tested in the following media conditions:
A. 100% unconditioned maintenance medium.
B. 100% unconditioned maintenance medium+10 ng/ml EGF.
C. 90% unconditioned maintenance medium/10% conditioned maintenance medium from cultured skin constructs between 10 and 12 days PAL.
D. 50% unconditioned maintenance medium/50% conditioned maintenance medium from cultured skin constructs between 10 and 12 days PAL.
E. 100% conditioned maintenance medium from cultured skin constructs between 10 and 12 days PAL.

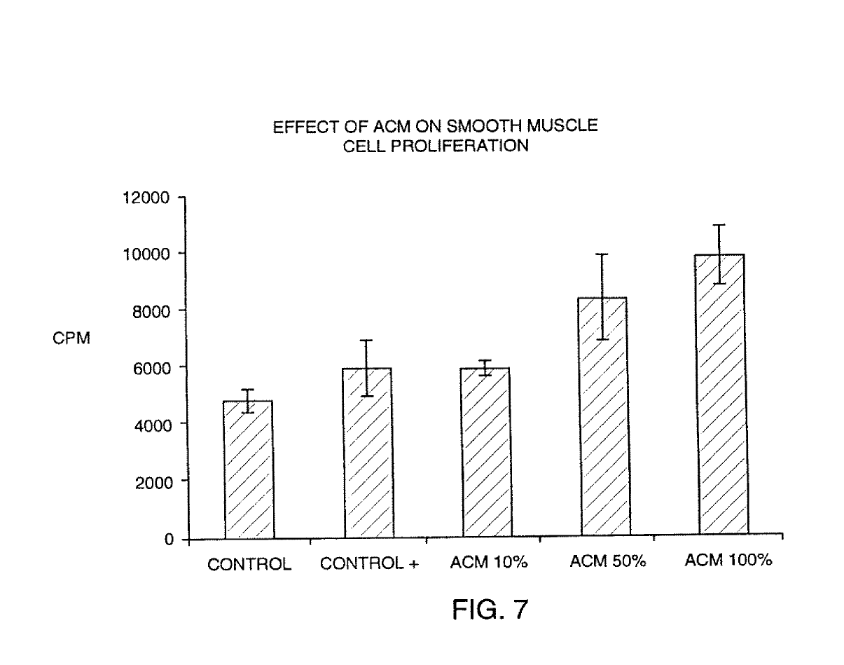

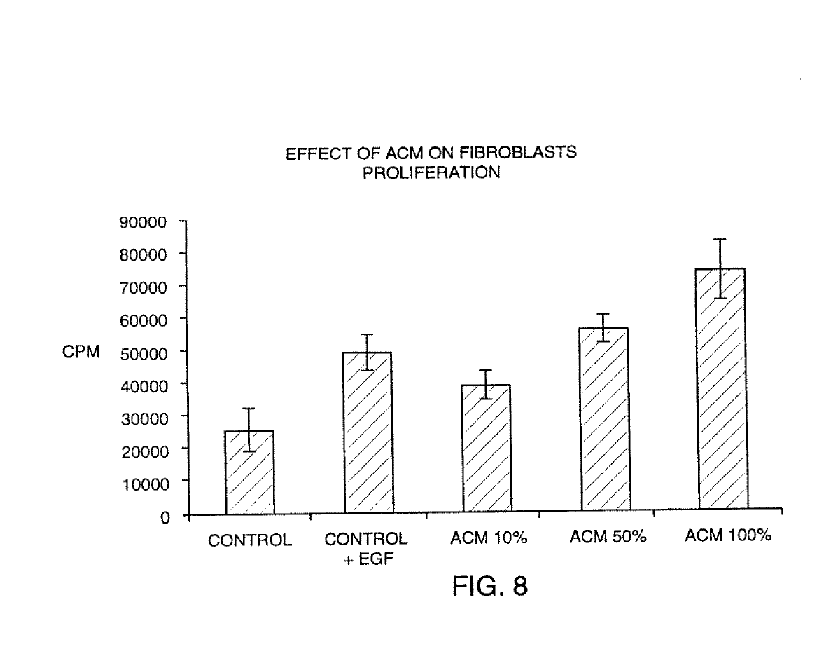

Results from the smooth muscle proliferation assay are presented in FIG. 7, and those from the dermal fibroblast proliferation assay are presented in FIG. 8. For these two cell types tested, conditioned medium from the cultured skin constructs stimulated cell proliferation at level above unconditioned media and are increasingly proliferative in cultures containing higher concentrations of conditioned medium. The findings suggest that cultured skin constructs produce cytokines that are biologically active with significant effects on cell proliferation.

### Example 8

### ELISA

Cytokines in conditioned media, unconditioned control media, the cotton pad used in airlift of the culture to bring it to the air-liquid interface, and cell extract obtained from the cultured skin construct were characterized using ELISA. Specifically, basic fibroblast growth factor (bFGF), keratinocyte growth factor (KGF), and transforming growth factor alpha (TGFα) were measured against control group cytokines: KGF (R&D Systems, cat. # DKG00); bFGF (R&D Systems, cat. # DFB00); and, TGFα (alpha) (Oncogene Research Products, cat. # QIA61).

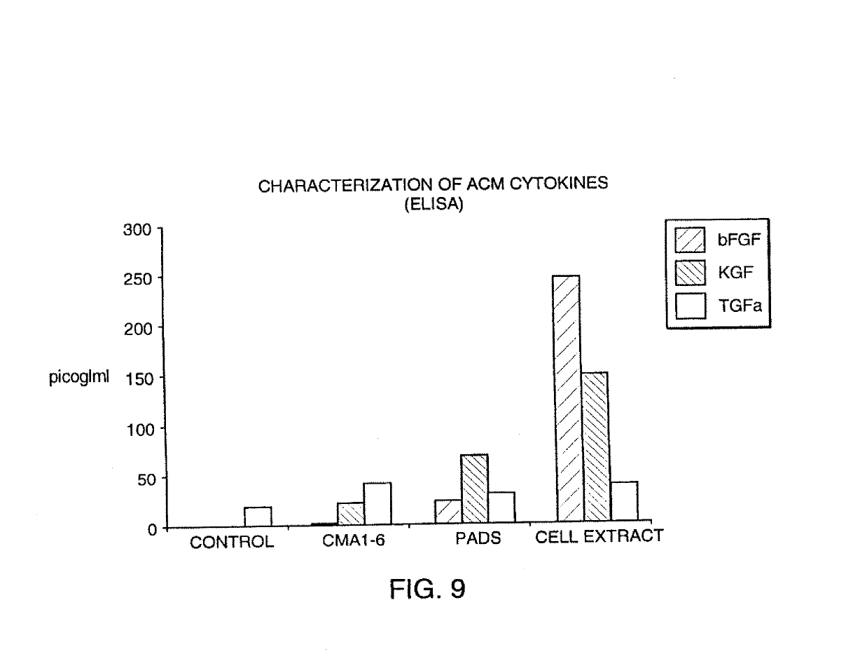

Results, as presented on FIG. 9, show that the small amounts of TGFα present in the fresh unconditioned medium while conditioned medium also contains KGF, bFGF, and increased levels of TGFα over that of control indicating that the cells of the cultured skin construct are producing these cytokines and depositing them into the medium as it is conditioned. The cotton pads from which conditioned medium is obtained by compressing the medium from it have even higher concentrations of bFGF and KGF and nearly the same amount of TGFα. The results also show that the cell extract obtained from the cultured skin construct contains high levels of all three components over the control medium and reemphasizes the use of cultured skin constructs for stimulation of wound healing processes.

### Example 9

### Purification/Concentration

Conditioned medium is filtered using ultrafiltration cell filters to remove large molecular weight components from the medium. The ultrafiltration is performed using the Amicon 8050 Ultrafiltration Cell product that contains an upper chamber and a lower chamber separated by a molecular weight cut-off filter. Conditioned medium is placed in the upper chambers of a number of ultrafiltration units and is forced through the filtration membrane using pressured nitrogen gas. The conditioned medium retentate containing is added to fresh unconditioned medium and added to keratinocyte cultures to test its proliferative ability when compared to fresh medium without the retentate. Cells cultured in fresh medium containing the conditioned medium filtrate exhibits increased proliferative ability over control cultures.

### Example 10

### The Effect of Conditioned Medium (ACM) on Human Keratinocyte Migration is Independent of the EGF-Receptor Pathway

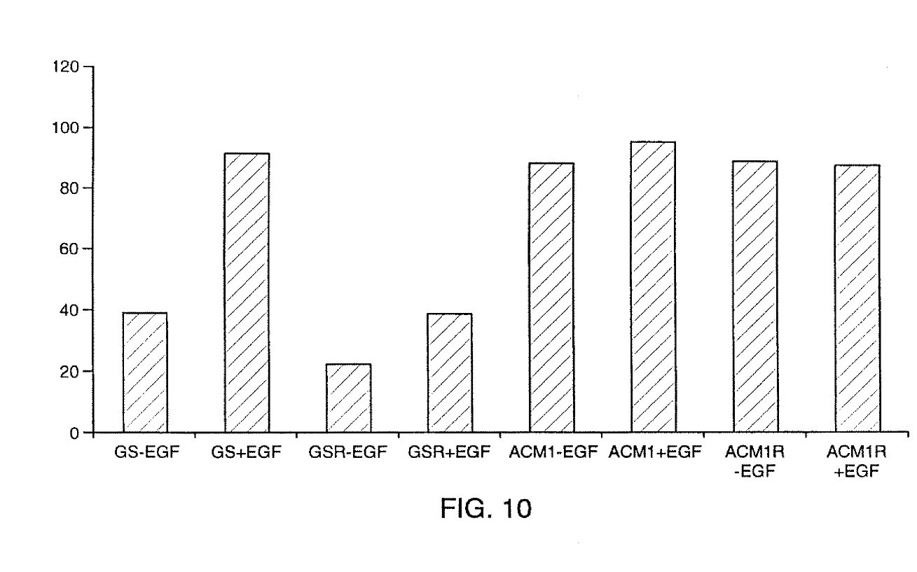

To test whether the migration effect caused by conditioned medium on keratinocyte cultures is due to EGF, the following experiment was conducted. Maintenance medium from Example 1 was collected and used individually because fresh maintenance medium does not initially contain EGF. Human keratinocytes were grown to confluence in 100 mm plates and then trypsinized to get a cell suspension. The cells were incubated ½ hour at room temperature, in 1 ml of medium in the presence (GSR or ACM1R) or in the absence (CS or ACM1) of a neutralizing anti-EGF receptor antibody (Upstate Biotechnology, catalog #05-101) at a concentration of 10 μg of antibody/10⁴ cells/ml of medium. Then, cells were plated on the fibrin substrate and tested for their ability to migrate as previously described in Example 6. Unconditioned (GS) or Conditioned mediums (ACM) were tested in presence or in absence of EGF (10 ng/ml). The results of the shown in FIG. 10 are representative of three experiments.

The present results show that cultured human keratinocytes produce one or more factors that stimulate migration of cultured human, this effect was comparable to that of EGF. However, the addition of EGF antibodies, which blocked the effect of EGF, did not abolish the effect of ACM suggesting that EGF is not responsible for the effects of the conditioned medium. These results emphasize the fact that ACM produce one or more factors other than EGF that greatly induce keratinocytes to migrate as it is needed for in vivo skin treatment.

Although the foregoing invention has been described in some detail by way of illustration and example for purposes of clarity and understanding, it will be obvious to one of skill in the art that certain changes and modifications may be practiced within the scope of the appended claims.

We claim:

1. A composition comprising conditioned cell medium containing one or more cultured skin agents synthesized and secreted from cultured skin cells including keratinocytes and/or fibroblasts for use as a pharmaceutical preparation or as a skin care product.

2. A method for producing a preparation containing a conditioned cell medium containing one or more cultured skin agents produced by cultured skin cells, comprising:
culturing skin cells in a nutrient containing medium to grow the skin cells and inducing skin cells to synthesize and secrete one or more cultured skin agents into the medium;
separating the conditioned medium containing one or more cultured skin agents from the cultured skin cells; and,
producing a preparation comprising the conditioned medium containing one or more cultured skin agents.

3. The method of claim 2, wherein the skin cell culture comprises keratinocytes.

4. The method of claim 2, wherein the skin culture comprises fibroblasts.

5. The method of claim 2, wherein the skin cell culture is a co-culture of keratinocytes and fibroblasts.

6. The use of a conditioned cell medium containing one or more cultured skin agents synthesized and secreted from cultural skin cells in the preparation of a pharmaceutical preparation.

7. A method of treating the skin an individual comprising:
applying topically to the skin a composition comprising a conditioned cell medium containing one or more cultured skin agents synthesized and secreted from cultured skin cells.

8. The method of claim 7, wherein the skin of the individual is treated to increase cell proliferation and generation.

9. The method of claim 7, wherein the skin of the individual is treated to decrease the rate of cell senescence in the skin.

10. A skin treatment comprising a topically applied composition for increasing cell proliferation and generation and for decreasing cell senescence formulated with conditioned cell medium containing one or more cultured skin agents synthesized and secreted from cultured skin cells.
